(19) United States
(12) Patent Application Publication
(10) Pub. No.: US 2008/0140151 A1
(43) Pub. Date: Jun. 12, 2008

(54) NERVE STIMULATION APPARATUS AND METHOD FOR THE TREATMENT OF HEAD PAIN

(76) Inventor: Jason A. Brodkey, Ann Arbor, MI (US)

Correspondence Address: GIFFORD, KRASS, SPRINKLE,ANDERSON & CITKOWSKI, P.C, PO BOX 7021, TROY, MI, 48007-7021, US

(21) Appl. No.: 11/608,990

(22) Filed: Dec. 11, 2006

(51) Int. Cl. A61N 1/05

(52) U.S. Cl. 607/46

(57) ABSTRACT

Nerve stimulation apparatus and methods particularly suited to the treatment of head pain (cephalgia) facilitate bilateral stimulation of the spine with a single protruding lead. The instrumentation includes an electrode assembly formed on/in a biocompatible substrate having a front surface, a back surface, a top edge and a bottom edge defining a height with a horizontal centerline, and opposing side edges defining a width greater than the height, the width being divided into right and left halves on either side of a vertical centerline. At least one first exposed electrode is disposed on the right half of the front surface, and at least one second exposed electrode is disposed on the left half of the front surface. The first and second electrodes are spaced apart by a distance of 1 centimeter or greater, preferably in the range of 1 to 3 centimeters. A pair of leads, one interconnected to a respective one of the first and second electrodes, protrude from the top or bottom edge of the substrate.

### FIELD OF THE INVENTION

This invention relates generally to nerve stimulation and, in particular, to nerve stimulation apparatus and methods particularly suited to the treatment of head pain (cephalgia).

### BACKGROUND OF THE INVENTION

Electrical stimulation of the spinal cord and the peripheral nerves (neurostimulation) are medical therapies used for pain relief or symptom relief from certain types of chronic pain and neurological disorders. While neurostimulation is not a cure for pain, the approach is often effective in reducing a patient's pain to a manageable level, enabling the patient to a more normal lifestyle.

Spinal cord and peripheral nerve stimulation typically use an implanted device to deliver low levels of electrical energy directly to nerve fibers. During a relatively short surgical procedure, one or more electrodes are placed in the space above of the spinal column (epidural space) and a stimulator unit is placed under the sldn (subdermally). Electrical leads connecting the stimulator unit to the electrodes are also routed subdermally. When the stimulator unit is turned on, electrical impulses are delivered to the electrodes, which stimulate nerve fibers associated with painful areas. The stimulation effectively replaces the pain messages with a more pleasant sensation called paresthesia.

Manufacturers offer a variety of leads to meet the needs of patients and physicians. Leads can vary by type (i.e., percutaneous or surgically implanted), the number of electrodes, electrode shape, configuration and spacing. Percutaneous leads can be implanted through a needle and may not need a surgical incision. As such they are faster and easier to place. Surgical or ‘paddle leads’ are larger and require a surgical incision. The advantages of surgical leads are that they are less prone to migration and their flat shape makes them more energy efficient. Percutaneous leads are almost always used for trial stimulation prior to permanent implantation.

Each lead terminates in a number of electrodes, and may contain as many as 16. The number of electrodes used depends upon the condition being treated as well as the physician's preference. For example, more complex pain patterns, such as those involving more than one area and more than one extremity (arms and/or legs), involve more nerve structures. Additional electrodes are often required to stimulate all of these structures.

FIG. 1 is a drawing that illustrates a stimulation lead sold by ANS (Advanced Neuromodulation Systems) of Plano, Tex. This particular lead, one of many different styles, uses two side-by-side sets of four commonly interconnected electrodes. That is, electrodes 102, 104, 106, 108 are connected to lead 110, whereas electrodes 112, 114, 116, 118 are connected to lead 120, The electrodes are disposed in a flexible, rubbery membrane.

To treat head pain, stimulation electrodes must be placed under the upper cervical vertebrae, usually under C1, C2 or C3. In practice, leads such as that illustrated in FIG. 1 are not optimized for this application. First, it is often advantageous to use separate, individually controlled electrodes on both side of the spine. This requires the use of two, separately placed electrodes of the type shown in the Figure. Secondly, since treatment for head pain may require placement under an adjacent vertebral structure, electrodes of the type shown in the Figure must be slid along the spinal cord so that the distalmost electrode(s) reach a target destination. However, since a row of electrodes are commonly connected, the proximal electrodes are activated in conjunction with the distal electrodes, resulting in wasted energy stimulating untargeted nerves. Often the distal, or inferior electrodes are not used at all and unnecessary surgical dissection is done in placing the leads. Unnecessary dissection in the spine adds to surgical risk.

### SUMMARY OF THE INVENTION

This invention resides in nerve stimulation apparatus and methods particularly suited to the treatment of head pain (cephalgia). The instrumentation includes an electrode assembly formed on/in a biocompatible substrate having a front surface, a back surface, a top edge and a bottom edge defining a height with a horizontal centerline, and opposing side edges defining a width greater than the height, the width being divided into right and left halves on either side of a vertical centerline. At least one first exposed electrode is disposed on the right half of the front surface, and at least one second exposed electrode is disposed on the left half of the front surface. The first and second electrodes are spaced apart by a distance of 1 centimeter or greater, preferably in the range of 1 to 3 cm. A pair of leads, one interconnected to a respective one of the first and second electrodes, protrude from the top or bottom edge of the substrate.

In the preferred embodiment, the biocompatible substrate is generally rectangular and flexible. The first and second electrodes are aligned on the horizontal centerline of the substrate with no other electrodes therebetween. The leads preferably protrude at or near the vertical centerline, resulting in a T-shaped structure. A tab extending from the top or bottom edge may be provided for fixation to a vertebral body.

In alternative embodiments, the electrode assembly includes two or more pairs of exposed electrodes on each side of the front surface, with associated interconnecting leads again protruding from the top or bottom edge of the substrate. One more electrodes on one side of the substrate may be larger than one or more other electrodes on that side. A pattern of exposed electrodes may be provided, one on each side of the front surface, arranged as mirror images about the vertical centerline of the substrate.

The apparatus may further include an electrical stimulator interconnected to the electrodes through the leads. With multiple electrodes on each side of the substrate, the stimulator is preferably operative to deliver positive and negative electrical potentials among different pairs of electrodes.

A method of neurostimulation comprises the steps of providing an electrode assembly according to the invention, surgically implanting the assembly against a spinal cord beneath a cervical vertebrae, and delivering electrical impulses to the electrodes to alleviate pain. For head pain, the electrodes are inserted beneath cervical vertebrae C1, C2, or C3.

### BRIEF DESCRIPTION OF THE DRAWINGS

FIG. 1 is a drawing of a typical prior-art electrical stimulation lead;

FIG. 2 is a drawing showing how a lead of the type shown in FIG. 1 may be positioned in an effort to treat head pain;

FIG. 3A is a drawing of an electrical stimulation lead according to the invention, optimized for head pain relief; and

FIG. 3B shows an alternative electrical stimulation lead according to the invention featuring opposing pairs of electrodes;

FIG. 3C shows an alternative electrical stimulation lead according to the invention featuring opposing pairs of electrodes with different sizes;

FIG. 3D shows an alternative electrical stimulation lead according to the invention featuring opposing multiple electrode patterns;

FIG. 4 is a drawing which shows one position of the electrode of FIG. 3 in place;

FIG. 5 is a top-down drawing that shows the way in which electrodes are placed according to the invention; and

FIG. 6 is a side-view drawing that shows the way in which electrodes according to the invention may be fastened to a vertebral body.

### DETAILED DESCRIPTION OF THE INVENTION

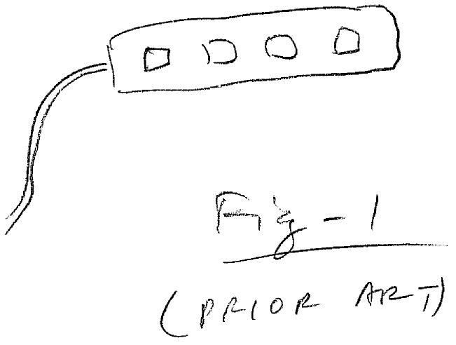

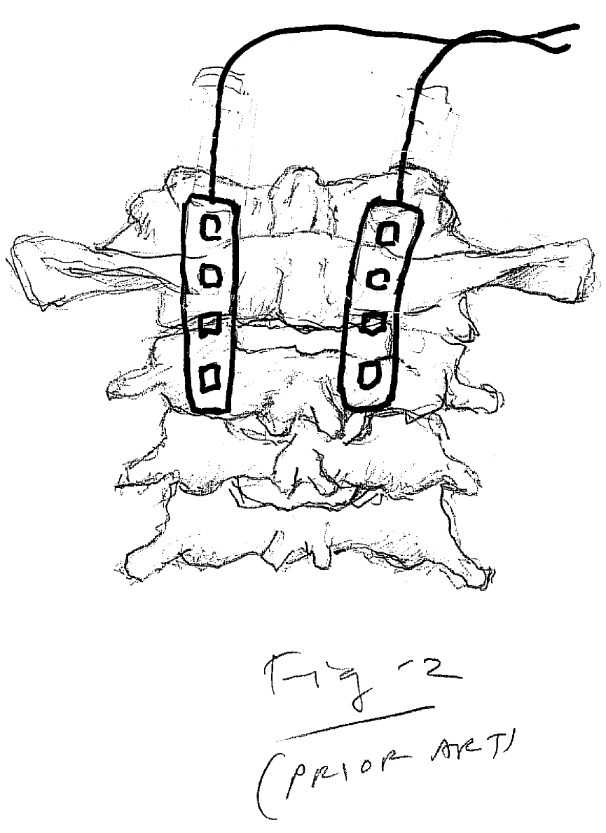

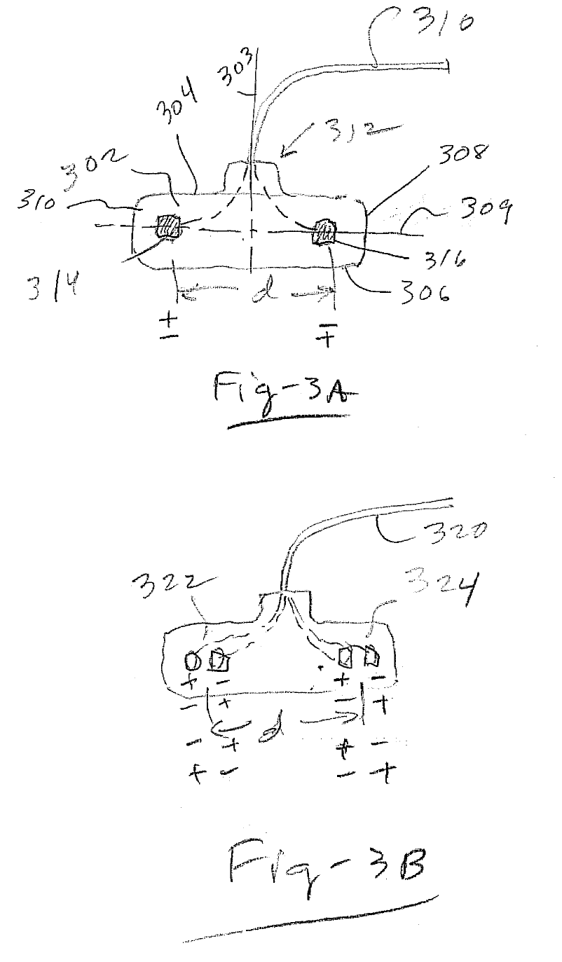

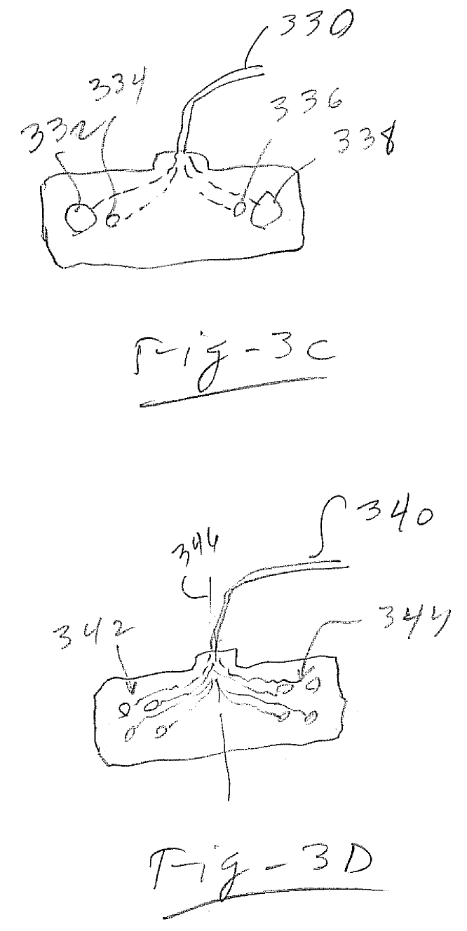

Having discussed the configuration and use of the prior-art electrode of FIG. 1, the reader's attention is turned to FIG. 3a, which shows a basic electrode assembly according to the invention optimized for the treatment of head pain. The assembly comprises a biocompatible substrate 302 having a top edge 304 and a bottom edge 306 defining a height, and opposing side edges 308, 310 defining a width greater than the height in the preferred embodiment. The basic shape is preferably rectangular overall, though elongate ovals and other geometries may alternatively be used, since the shape of the substrate is not as critical as the electrode spacing, d, which is one centimeter or more, preferably in the range of 1 to 3 centimeters.

The substrate 302 is divided by a vertical centerline 303 into right and left halves, with electrodes 304, 306 being symmetrically disposed in the right and left halves, preferably along a horizontal centerline 309. The lead 310 includes two conductors in this case, one going to electrode 314, and the other going to electrode 316. In the preferred embodiment, the lead 310 protrudes from the top or bottom edge of the substrate along vertical centerline 303, which divides the assembly into right and left halves. As shown by the symbols on the drawing, the electrodes are preferably operated with consecutively switching polarities (±, ±, etc.) thereby providing stimulation across the target area using a single structure as opposed to the current need for two electrodes.

FIG. 3b is a drawing which shows an alternative embodiment of the invention, wherein each half of the substrate includes a pair of electrodes, 322, 324. Lead 320 from a stimulator unit, includes conductors interconnected to each one of the electrodes, which are generally spaced apart by a distance of 1 to 4 millimeters overall. This and other embodiments facilitate any type of appropriate electrical stimulation to be delivered, including positive and negative excursions on either or both sides of the assembly, indicated by the plus and minus signs.

The electrode assembly of FIG. 3c is a derivative of that depicted in FIG. 3b, but wherein one of the electrodes of each pair, 332, 338, are larger than the other electrodes on that side, 334, 336. As opposed to the outermost electrodes being larger than the innermost, a reverse situation is also anticipated. Patterns with multiple electrodes on either side are also not precluded, as shown in FIG. 3d. These patterns, such as four on each side, 342, 344, would preferably be mirror images about the vertical centerline 346. Again, lead assembly 340, preferably protruding at or near the vertical centerline 346, would include sufficient electrical conductors to make independent contact to all of the various electrodes, shown with the broken lines.

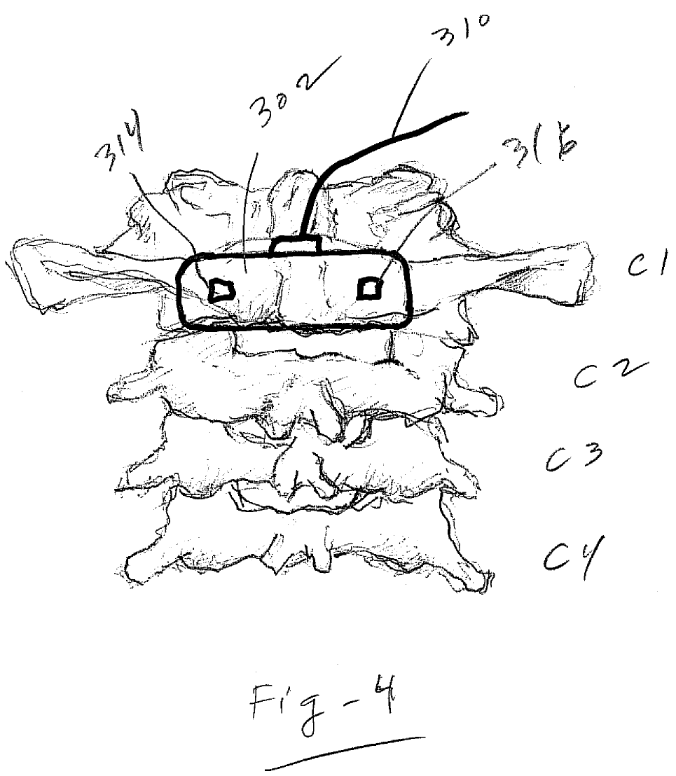

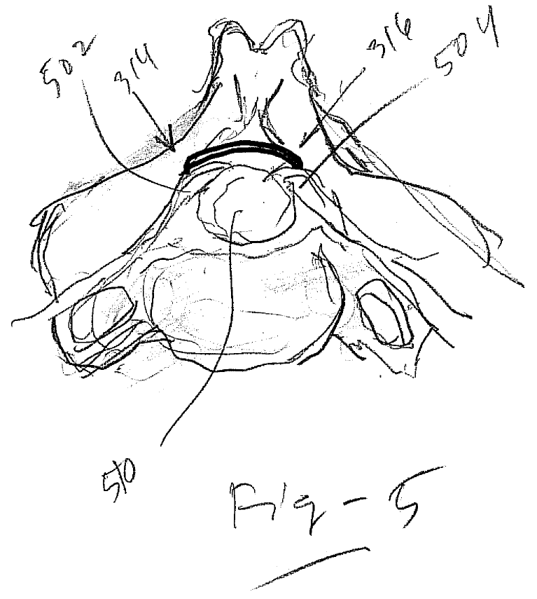

FIG. 4 is a drawing which shows a preferred placement of the electrode assembly of FIG. 3a, with the understanding that the alternative electrode assembly configurations could be similarly placed. As shown in the drawing, the configuration of the electrode assembly allows it to be laterally placed over the spinal cord, with the electrodes 314, 316 directly over the nerve roots responsible for head pain. The various levels are indicated with C1, C2, C3 corresponding to first cervical, etc.

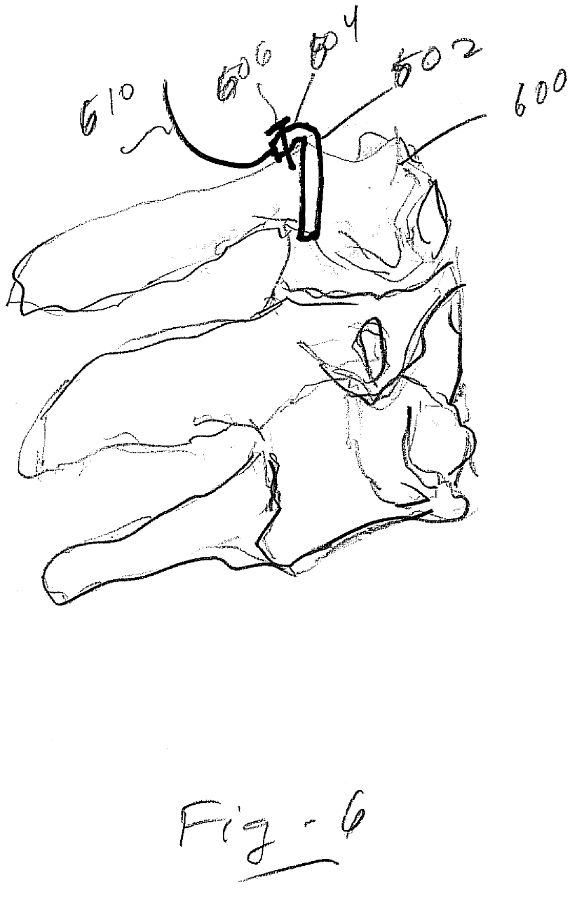

FIG. 5 is a top-down drawing which shows the electrode assembly placement of FIG. 4, with electrodes 314, 316 being directly over nerve roots 502, 504, which emanate from the spinal cord 510. FIG. 6 is a drawing which shows the way in which a tab 604 on an electrode assembly 602, may be stapled at 606 to an appropriate vertebral body 600. The leads protruding from the electrode assembly are indicated at 610.

I claim:

1. Nerve stimulation apparatus, comprising:
an electrode assembly, including:
a biocompatible substrate having a front surface, a back surface, a top edge and a bottom edge defining a height with a horizontal centerline, and opposing side edges defining a width greater than the height, the width being divided into right and left halves on either side of a vertical centerline;
a first exposed electrode on the right half of the front surface;
a second exposed electrode on the left half of the front surface;
the first and second electrodes being spaced apart by a distance of 1 centimeter or greater; and
a pair of leads, one interconnected to a respective one of the first and second electrodes, the leads protruding from the top or bottom edge of the substrate.

2. The nerve stimulation apparatus of claim 1, wherein the biocompatible substrate is flexible.

3. The nerve stimulation apparatus of claim 1, wherein the biocompatible substrate is generally rectangular.

4. The nerve stimulation apparatus of claim 1, wherein the leads protrude at or near the vertical centerline.

5. The nerve stimulation apparatus of claim 1, further including a tab extending from the top or bottom edge adapted for fixation to a vertebral body.

6. The nerve stimulation apparatus of claim 1, wherein the first and second electrodes are aligned on the horizontal centerline.

7. The nerve stimulation apparatus of claim 1, wherein there are no other electrodes between the first and second electrodes.

8. The nerve stimulation apparatus of claim 1, further including:
two pairs of exposed electrodes, one pair on each side of the front surface; and
leads interconnected to all of the electrodes, the leads protruding from the top or bottom edge of the substrate.

10. The nerve stimulation apparatus of claim 1, further including:
two pairs of exposed electrodes, one pair on each side of the front surface;
leads interconnected to all of the electrodes, the leads protruding from the top or bottom edge of the substrate; and
wherein one electrode of each pair is larger than the other electrode of that pair.

11. The nerve stimulation apparatus of claim 17 further including: a pattern of exposed electrodes, one pattern on each side of the front surface;
leads interconnected to all of the electrodes, the leads protruding from the top or bottom edge of the substrate; and
wherein the patterns are mirror images about the vertical centerline.

12. The nerve stimulation apparatus of claim 1, further including an electrical stimulator interconnected to the electrodes through the leads.

13. The nerve stimulation apparatus of claim 10, further including an electrical stimulator interconnected to the electrodes through the leads.

14. The nerve stimulation apparatus of claim 10, further including:
an electrical stimulator interconnected to the electrodes through the leads; and
wherein the stimulator is operative to deliver positive and negative electrical potentials to the electrodes comprising each pair.

15. A method of neurostimulation, comprising the steps of: providing the electrode assembly of claim 1; and
surgically implanting the assembly against a spinal cord beneath a cervical vertebrae.

16. A method of neurostimulation, comprising the steps of:
providing the electrode assembly of claim 1; and
surgically implanting the assembly against a spinal cord beneath a cervical vertebrae C1, C2, or C3.

17. A method of neurostimulation, comprising the steps of:
providing the electrode assembly of claim 10; and
surgically implanting the assembly against a spinal cord beneath a cervical vertebrae; and
operating the stimulator to deliver positive and negative electrical potentials to each pair of electrodes.
